(19) United States
(12) Patent Application Publication
(10) Pub. No.: US 2024/0350442 A1
(43) Pub. Date: Oct. 24, 2024

(54) METHODS OF TREATING CANCER

(71) Applicant: Inspirna, Inc., Long Island City, NY (US)

(72) Inventors: Masoud Fakhr TAVAZOIE, New York, NY (US); David M. DARST, JR., New York, NY (US); Foster Casimir GONSALVES, Long Island City, NY (US); Isabel KURTH, New York, NY (US)

(21) Appl. No.: 18/653,106

(22) Filed: May 2, 2024

Related U.S. Application Data
(63) Continuation of application No. 17/119,661, filed on Dec. 11, 2020, now Pat. No. 12,011,427.
(60) Provisional application No. 62/946,581, filed on Dec. 11, 2019.

(51) Int. Cl. A61K 31/195; A61K 9/00; A61K 31/4375; A61K 31/513; A61K 31/519; A61P 35/04

(52) U.S. Cl. CPC A61K 31/195; A61K 9/0019; A61K 31/4375; A61K 31/513; A61K 31/519; A61P 35/04

(57) ABSTRACT

The invention features methods of treating cancer with β-GPA. The disclosure also provides methods of treating cancer including combinations of β-GPA and additional anti-cancer therapies.

### BACKGROUND

β-Guanidinopropionic acid (β-GPA), also referred to as guanidinopropionic acid, beta-guanidinopropionic acid or, N-(aminoiminomethyl)-beta-alanine is a creatine analog. Studies on animals (rats, monkeys, hamsters) show that acidic guanidine derivatives such as β-GPA can ameliorate hyperglycemia in animal models of noninsulin-dependent diabetes. Accordingly, it is sometimes used as a dietary supplement in diabetic patients to regulate blood sugar levels.

β-GPA has recently been found to be effective for the suppression of metastasis, particularly liver metastasis in gastrointestinal cancers, e.g., see International Patent Publication WO2014/071067. Accordingly, the development of dosing regimens of β-GPA which result in efficacy while reducing adverse events for the treatment of cancer are needed.

### SUMMARY OF THE INVENTION

The invention features methods of treating cancer by administering about 1,500 mg to about 4,000 mg of β-GPA twice daily. The inventors have discovered that this dosing regimen surprisingly results in higher than expected levels of systemically circulating β-GPA.

Accordingly, in one aspect, the invention features a method of treating cancer (e.g., gastrointestinal cancer such as colon cancer or gastric cancer, pancreatic cancer, liver cancer, breast cancer, prostate cancer, lung cancer, adenocarcinoma of the esophagogastric junction, and melanoma) in a subject in need thereof. This method includes administering about 1,500 mg to about 4,000 mg (e.g., about 1,500 mg to about 2,000 mg, about 1,750 mg to about 2,250 mg, about 2,000 mg to about 2,500 mg, about 2,250 mg to about 2,750 mg, about 2,400 mg to about 2,800 mg, about 2,700 mg to about 3,000 mg, about 2,750 mg to about 3,250 mg, about 3,100 mg to about 3,400 mg, about 3,200 mg to about 3,600 mg) of β-GPA, or a pharmaceutically acceptable salt thereof to the subject twice per day. In some embodiments of any of the foregoing methods, the method includes administering between about 2,400 mg and about 3,600 mg of β-GPA, or a pharmaceutically acceptable salt thereof to the subject twice per day. In some embodiments of any of the foregoing methods, the method includes administering about 2,400 mg of β-GPA, or a pharmaceutically acceptable salt thereof to the subject twice per day. In some embodiments of any of the foregoing methods, the method includes administering about 3,600 mg of β-GPA, or a pharmaceutically acceptable salt thereof to the subject twice per day.

In some embodiments of any of the foregoing methods, the method further includes administering one or more further anti-cancer therapies (e.g., radiation therapy, surgery, and/or one or more therapeutic agents). In some embodiments of any of the foregoing methods, the one or more further anti-cancer therapies includes folinic acid, fluorouracil, irinotecan, and/or oxaliplatin. In some embodiments of any of the foregoing methods, the one or more further anti-cancer therapies includes FOLFIRI, i.e., folinic acid, fluorouracil, and irinotecan. In some embodiments of any of the foregoing methods, the method includes administering about 180 mg/m² of irinotecan intravenously over 90 minutes concurrently with about 400 mg/m² or 2×250 mg/m² folinic acid intravenously over 120 minutes followed by an optional about 400-500 mg/m² (e.g., about 400 mg/m²) bolus of fluorouracil intravenously followed by an about 2400-3000 mg/m² (e.g., about 2400 mg/m²) infusion of fluorouracil intravenously over 46 hours on days 1 and 15 of each 28-day cycle, e.g., repeated about every fourteen days. In some embodiments of any of the foregoing methods, the one or more therapeutic agents is cyclocreatine, a RNAi agent, a nucleic acid, a vector, 5-fluorouracil, oxaliplatin, irinotecan, capecitabine, gemcitabine, cetuximab, taxol, avastin, folinic acid (leucovorin), regorafenib, zaltrap, topoisomerase I inhibitors, etirinotecan pegol, tivantinib, sonolisib, sorafenib, linifanib, kinase inhibitors, telatinib, BMS-908662 (i.e., methyl/V-[6-[2-(5-chloro-2-methylphenyl)-1-hydroxy-3-oxoisoindol-1-yl]-1H-benzimidazol-2-yl]carbamate), robatumumab, and/or IGF1-R inhibitors.

In some embodiments of any of the foregoing methods, the method further includes surgery (e.g., prior to, or subsequent to, administration of β-GPA, or a pharmaceutically acceptable salt thereof).

In some embodiments of any of the foregoing methods, the cancer is metastatic cancer (e.g., metastatic gastrointestinal cancer such as metastatic colon cancer or metastatic gastric cancer, metastatic pancreatic cancer, metastatic liver cancer, metastatic breast cancer, metastatic prostate cancer, metastatic lung cancer, metastatic adenocarcinoma of the esophagogastric junction, or metastatic melanoma).

In some embodiments of any of the foregoing methods, the cancer is gastrointestinal cancer (e.g., colorectal cancer, gastric cancer, or adenocarcinoma of the esophagogastric junction).

In some embodiments of any of the foregoing methods, the cancer expresses CKB. In some embodiments of any of the foregoing methods, the subject is identified to have, or to be at risk of having, metastatic cancer (e.g., on the basis of the expression level of CKB being above a predetermined reference value).

In some embodiments of any of the foregoing methods, the cancer is resistant to one or more therapeutic agents. In some embodiments of any of the foregoing methods, the cancer progressed on or after treatment with one or more anti-cancer therapies.

In some embodiments of any of the foregoing methods, the β-GPA, or a pharmaceutically acceptable salt thereof is the succinate salt of β-GPA (e.g., the 2:1 succinate salt of β-GPA).

### Definitions

As used herein, the term “about” represents a value that is in the range of +10% of the value that follows the term “about.”

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

The term “cancer” refers to any cancer caused by the proliferation of malignant neoplastic cells, such as tumors, neoplasms, carcinomas, sarcomas, leukemias, and lymphomas.

A cancer “determined to be drug resistant,” as used herein, refers to a cancer that is drug resistant, based on unresponsiveness or decreased responsiveness to a chemotherapeutic agent, or is predicted to be drug resistant based on a prognostic assay (e.g., a gene expression assay).

By a “drug resistant” cancer is meant a cancer that does not respond, or exhibits a decreased response to, one or more chemotherapeutic agents (e.g., any agent described herein).

As used herein, the term “failed to respond to a prior therapy” or “refractory to a prior therapy,” refers to a cancer that progressed despite treatment with the therapy.

As used herein, “metastatic tumor” refers to a tumor or cancer in which the cancer cells forming the tumor have a high potential to or have begun to, metastasize, or spread from one location to another location or locations within a subject, via the lymphatic system or via hematogenous spread, for example, creating secondary tumors within the subject. Such metastatic behavior may be indicative of malignant tumors. In some cases, metastatic behavior may be associated with an increase in cell migration and/or invasion behavior of the tumor cells.

Examples of cancers that can be defined as metastatic include but are not limited to non-small cell lung cancer (e.g., non-squamous non-small cell lung cancer), breast cancer, ovarian cancer, colorectal cancer, biliary tract cancer, bladder cancer, brain cancer including glioblastomas and medulloblastomas, cervical cancer, choriocarcinoma, endometrial cancer, esophageal cancer, gastric cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

As used herein, the term “pharmaceutical composition” refers to an active compound, formulated together with one or more pharmaceutically acceptable carriers. In some embodiments, active compound is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Those of ordinary skill in the art are familiar with a variety of agents and materials useful as excipients.

The term “pharmaceutically acceptable salt,” as use herein, refers to those salts of the compounds described here that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art.

The term “subject,” as used herein, refers to a human or non-human animal (e.g., a mammal such as a non-human primate, horse, cow, or dog).

The term “treatment” (also “treat” or “treating”), in its broadest sense, refers to any administration of a substance (e.g., provided compositions) that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively, or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### DETAILED DESCRIPTION OF THE INVENTION

The invention features methods of treating cancer by administering about 2,000 mg to about 4,000 mg of β-GPA twice daily. The inventors have discovered that this dosing regimen surprisingly results in higher than expected levels of circulating β-GPA.

### β-GPA

β-GPA has the structure:

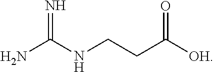

β-GPA is zwitterionic and highly soluble in water (>50 mg/mL), but has low solubility in organic solvents. β-GPA possesses a basic guanidino group, and is thus capable of forming both 1:1 (β-GPA:acid) and 2:1 (β-GPA:acid) salts with diacids. As used herein, a “2:1 salt” of β-GPA with a diacid, e.g., a 2:1 succinate salt, refers to a salt including two molecules of β-GPA and one molecule of the diacid, e.g., a “2:1 succinate salt” includes two molecules of β-GPA and one molecule of succinic acid.

### Treatment Methods

β-GPA has recently been found to be effective for the suppression of metastasis. The mechanism of action has been hypothesized as inhibition of creatine transport and/or creatine kinase. The phosphocreatine system promotes metastasis by enhancing the survival of disseminated cancer cells in the liver by acting as an energetic store for ATP generation to endure hepatic hypoxia. Inhibition of creatine transport into cancer cells limits the amount of phosphocreatine available to use in the production of ATP. Inhibition of creatine kinase inhibits the production of ATP through conversion of phosphocreatine to creatine.

Typical vascularized tumors that can be treated with the methods of the invention include solid tumors, particularly carcinomas, which require a vascular component for the provision of oxygen and nutrients. Exemplary solid tumors include, but are not limited to, carcinomas of the lung, breast, bone, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, glioblastomas, neuroblastomas, Kaposi's sarcoma, and sarcomas.

Treating cancer can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a method of the invention.

### Compositions

Within the scope of this invention is a composition that contains a suitable excipient and one or more of the pharmaceutically acceptable salts described above. The composition can be a pharmaceutical composition that contains a pharmaceutically acceptable excipient, a dietary composition that contains a dietarily acceptable suitable excipient, or a cosmetic composition that contains a cosmetically acceptable excipient.

The term “pharmaceutical composition” refers to the combination of an active agent with a excipient, inert or active, making the composition especially suitable for diagnostic or therapeutic use in vivo or ex vivo. A “pharmaceutically acceptable excipient,” after administered to or upon a subject, does not cause undesirable physiological effects. The excipient in the pharmaceutical composition must be “acceptable” also in the sense that it is compatible with the active ingredient and can be capable of stabilizing it. One or more solubilizing agents can be utilized as pharmaceutical excipients for delivery of an active compound. Examples of a pharmaceutically acceptable excipient include, but are not limited to, biocompatible vehicles, adjuvants, additives, and diluents to achieve a composition usable as a dosage form.

As described above, the pharmaceutical compositions of the present invention additionally include a pharmaceutically acceptable excipient, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention.

The above-described composition, in any of the forms described above, can be used for treating cancer, or any other disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. In addition, fixed oils are conventionally employed as a solvent or suspending medium (e.g., synthetic mono- or diglycerides). Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets, emulsions and aqueous suspensions, dispersions, and solutions. When aqueous suspensions or emulsions are administered orally, the active ingredient can be suspended or dissolved in an oily phase combined with emulsifying or suspending agents. If desired, certain sweetening, flavoring, or coloring agents can be added.

### Combination Therapies

In some embodiments, the pharmaceutical composition may further include an additional compound having antiproliferative activity. It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

### EXAMPLES

### Example 1. β-GPA Pharmacokinetics

Method: Subjects were administered RGX-202 (a highly compressible salt form of β-GPA) in a regimen of 600 mg BID, 1,200 mg BID, 2,400 mg BID, or 3,600 mg BID, and plasma samples were taken from the subjects and tested using the protocol described below for levels of β-GPA over the course of 24 hours after administration.

The analyte, β-GPA, and internal standard (IS), [13C415N3]-β-GPA, are extracted from 50.0 μL of human plasma by a protein precipitation extraction procedure. The extraction procedure begins with the addition of 50.0 μL of internal standard working solution to all wells except double blanks, which receive 50.0 μL of water. Next the plate is covered and vortexed. Then 500 μL of acetonitrile/methanol (50/50, v/v) is added to all wells. Next the plate is covered, vortexed and centrifuged. Using a Tomtec Quadra 4, 200 μL of the supernatant is transferred into a new plate. Then 300 μL acetonitrile/methanol (50/50, v/v) is added to all wells. The plate is then sealed and vortexed. The extracts are chromatographed under reverse phase conditions on a Luna HPLC column 50×2.0 mm, 3 μm column using a gradient system with 10 mM ammonium acetate in water and acetonitrile. The compounds are detected and quantified by tandem mass spectrometry in positive ion mode on a MDS Sciex API 4000 equipped with a Turbo Ionspray® interface.

Results: As shown (by dose normalized accumulation ratios) in Table 1 below, administration with 2,400 mg BID or 3,600 mg BID of β-GPA results in higher than expected AUC and Cₘₐₓ levels in the subjects.

TABLE 1

| | | | Dose- | | | Dose | |
|---|---|---|---|---|---|---|---|
| | | | Normalized | AUC | | Normalized | Cₘₐₓ |
| | | Average | Average | Dose | Average | Average | Dose |
| | | AUC₀₋₂₄ | AUC₀₋₂₄ | Acc. | Cₘₐₓ | Cₘₐₓ | Acc. |
| Dose | | (ng-hr/mL) | (ng-hr/mL) | Ratio | (ng/mL) | (ng/mL) | Ratio |
| 600 | mg BID | 15,700 | 13.1 | 1.0 | 1,400 | 1.2 | 1.0 |
| 1,200 | mg BID | 45,200 | 18.8 | 1.4 | 4,790 | 2.0 | 1.7 |
| 2,400 | mg BID | 164,800 | 34.3 | 2.7 | 26,800 | 5.6 | 4.8 |
| 3,600 | mg BID | 241,097 | 33.5 | 2.6 | 47,660 | 6.6 | 5.7 |

### Example 2. β-GPA for the Treatment of GI Tumors

Method: Subjects with advanced gastrointestinal tumors (e.g., locally advanced and unresectable, or metastatic) were administered multiple doses of orally administered RGX-202 as a monotherapy or in combination with irinotecan, folinic acid, and fluorouracil. In the monotherapy arm of the study, RGX-202 was administered orally twice or three times daily on days 1-28 of each 28-day cycle. The dose regimen was dependent on the cohort in which the patient was enrolled. In the combination arm of the study, RGX-202 was administered in the same way as described for the monotherapy arm in combination with FOLFIRI. FOLFIRI was administered by intravenous administration of irinotecan (180 mg/m²) over 90 minutes concurrently with intravenous administration of folinic acid (400 mg/m²) over 2 hours, followed by fluorouracil (5-FU) (400 mg/m²) intravenous bolus and then 5-FU (2400 mg/m²) intravenous infusion over 46 hours, on days 1 and 15 of each 28-day cycle.

During the dose escalation, subjects were evaluated for pharmacokinetics (using the method as described in Example 1), pharmacodynamics, safety, and efficacy of the drug.

Results: The subjects did not exhibit dose limiting toxicity. Objective monotherapy (RGX-202) and combination therapy (RGX-202+FOLFIRI) activities were observed. Of the 7 subjects who received the combination therapy, 6 subjects showed stable disease (as described by RECIST 1.1 guidelines) after 40 weeks of treatment (e.g., doses of RGX-202≥1,800 mg BID). Of the 10 subjects who received the monotherapy, one subject exhibited a partial response (at a dose of 3,600 mg BID after 40 weeks), and three exhibited stable disease (at doses of 1,200 mg BID, 2,400 mg BID, and 3,600 mg BID).

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth. This application claims the benefit of U.S. provisional Ser. No. 62/946,581, filed Dec. 11, 2019, which is incorporated herein in its entirety.

1. A method of treating cancer in a subject in need thereof, the method comprising orally administering about 1500 mg to about 4000 mg of β-guanidinopropionic acid (β-GPA), or a pharmaceutically acceptable salt thereof to the subject, twice per day.

2. The method of claim 1, wherein the method comprises administering about 2400 mg to about 4000 mg of β-GPA, or a pharmaceutically acceptable salt thereof, to the subject, twice per day.

3. The method of claim 2, wherein the method comprises administering about 2750 mg to about 3250 mg of β-GPA, or a pharmaceutically acceptable salt thereof, to the subject, twice per day.

4. The method of claim 1, wherein the method comprises administering about 2400 mg of β-GPA, or a pharmaceutically acceptable salt thereof to the subject, twice per day.

5. The method of claim 1, wherein the method comprises administering about 3600 mg of β-GPA, or a pharmaceutically acceptable salt thereof to the subject, twice per day.

6. The method of claim 1, wherein the method comprises administering β-GPA, or a pharmaceutically acceptable salt thereof to the subject, twice per day, in an amount selected from the group consisting of about 1500 mg, about 1750, about 2250, about 2400 mg, about 2500, about 2600 mg, about 2750 mg, 2800 mg, about 3100, about 3200, about 3400, about 3600, and about 4000 mg.

7. The method of claim 1, wherein the method further comprises administering one or more further anti-cancer therapies.

8. The method of claim 7, wherein the one or more anti-cancer therapies comprise radiation therapy, surgery, and/or one or more therapeutic agents.

9. The method of claim 7, wherein the one or more further anti-cancer therapies comprise folinic acid, fluorouracil, irinotecan, and/or oxaliplatin.

10. The method of claim 7, wherein the one or more further anti-cancer therapies comprise folinic acid, fluorouracil, and irinotecan.

11. The method of claim 10, wherein the method comprises administering about 180 mg/m² of irinotecan intravenously over 90 minutes concurrently with about 400 mg/m² folinic acid intravenously over 120 minutes, followed by an about 2400 mg/m² infusion of fluorouracil intravenously over about 46 hours.

12. The method of claim 11, wherein the administering is repeated about every fourteen days.

13. The method of claim 7, wherein the one or more further anti-cancer therapies comprises bevacizumab.

14. The method of claim 7, wherein the method further comprises surgery.

15. The method of claim 14, wherein the surgery is prior to administration of β-GPA, or a pharmaceutically acceptable salt thereof.

16. The method of claim 1, wherein the cancer is a gastrointestinal cancer.

17. The method of claim 16, wherein the gastrointestinal cancer is colorectal cancer, gastric cancer, or adenocarcinoma of the esophagogastric junction.

18. The method of claim 17, wherein the gastrointestinal cancer is colorectal cancer.

19. The method of claim 1, wherein the cancer is locally advanced and unresectable, or metastatic cancer.

20. The method of claim 1, wherein the cancer expresses creatine kinase B (CKB).

21. The method of claim 1, wherein the cancer is resistant to one or more therapeutic agents.

22. The method of claim 1, wherein the cancer progressed on or after treatment with one or more anti-cancer therapies.

23. The method of claim 1, wherein the β-GPA, or a pharmaceutically acceptable salt thereof is a succinate salt of β-GPA.

24. The method of claim 23, wherein the succinate salt of β-GPA is a 2:1 succinate salt of β-GPA.

25. The method of claim 1, wherein the method comprises treating colorectal cancer in a subject in need thereof, the method comprising:
(a) orally administering about 2400 mg to about 3600 mg of a 2:1 succinate salt of β-GPA, or a pharmaceutically acceptable salt thereof to the subject, twice per day, and
(b) administering folinic acid, fluorouracil, irinotecan, and bevacizumab to the subject.
